(19) United States
(12) Patent Application Publication
(10) Pub. No.: US 2026/0201368 A1
(43) Pub. Date: Jul. 16, 2026

(54) SYSTEM AND METHOD FOR TOTAL NUCLEIC ACID LIBRARY PREPARATION VIA TEMPLATE-SWITCHING

(71) Applicant: Kapa Biosystems, Inc., Wilmington, MA (US)

(72) Inventors: Nicolette Adams, Cape Town (ZA); Martin Ranik, Dublin, CA (US); Ruben Gerhard van der Merwe, Cape Town (ZA); Eric van der Walt, Cape Town (ZA); Johan Christiaan Visser, Cape Town (ZA); Ross Iain McAllister Wadsworth, Cape Town (ZA)

(21) Appl. No.: 19/135,021

(22) PCT Filed: Dec. 7, 2023
(86) PCT No.: PCT/EP2023/084672

§ 371 Date: Jun. 2, 2025

Related U.S. Application Data
(60) Provisional application No. 63/386,725, filed on Dec. 9, 2022.

(51) Int. Cl. C12N 15/10; C12N 9/12; C40B 40/06; C40B 50/06

(52) U.S. Cl. CPC C12N 15/1096; C12N 9/1276; C12N 15/1093; C12Y 207/07049; C40B 40/06; C40B 50/06

(57) ABSTRACT

The present disclosure provides a method for carrying out a template-switching reaction on a nucleic acid sample including at least one double-stranded DNA and at least one RNA. The method includes performing a first template-switching reaction on the nucleic acid sample in the absence of at least one dNTP selected from dATP, dCTP, dGTP, and dTTP, thereby forming a first nucleic acid product comprising the double-stranded DNA having at least one extended 3′ end complementary to a first template-switch oligonucleotide. The method further includes performing a second template-switching reaction on the nucleic acid sample, thereby forming a second nucleic acid product comprising a first primer extension product complementary to at least a portion of the RNA, the first primer extension product having an extended 3′ end complementary to the second template-switch oligonucleotide.

### CROSS-REFERENCE TO RELATED APPLICATIONS

This patent application claims priority to U.S. Provisional Patent Application No. 63/386,725 , filed Dec. 9, 2022, which is incorporated herein by reference in its entirety.

### STATEMENT REGARDING FEDERALLY SPONSORED RESEARCH

Not applicable.

### BACKGROUND

The disclosure relates, in general, to library preparation for next generation sequencing of nucleic acids and, more particularly, to a system and method for total nucleic acid library preparation and targeted sequencing via template-switching.

In order to analyze nucleic acid samples using existing sequencing techniques, it is generally necessary to first prepare and optionally enrich the nucleic acids in the sample using one or more library preparation schemes, target enrichment schemes, or a combination thereof. Library preparation schemes are often employed to render a nucleic acid sample compatible with a given sequencing technology, for example, through the addition of common nucleic acid adapter sequences to the ends nucleic acid fragments derived from the sample. By comparison, target enrichment schemes are often employed for the selective isolation of specific genomic regions of interest prior to sequencing. Such enrichment methods are suited for experiments in which it may be desirable to study less than the entirety of the nucleic acid sequences derived from a biological source, but more than just a few (e.g., more than 1000) of the nucleic acid sequences.

In one aspect, it can be advantageous to generate both RNA and DNA sequencing libraries from the sample; however, existing library preparation and target enrichment schemes are, in general, not broadly applicable for different types of nucleic acids. For example, a particular scheme may be applicable for the preparation of a library starting with either DNA or RNA, but not both. Moreover, in the case it is desirable to prepare a nucleic acid library from both DNA and RNA derived from the same sample, additional steps may be required to first separate the DNA from the RNA for individual processing.

Previous approaches have provided limited solutions for the integration of DNA and RNA library generation. For example, US Patent Application No. 2018/0080021 to Reuter et al. describes a method for the simultaneous sequencing of RNA and DNA from the same sample. The approach taught by Reuter et al. is based on the use of i) Tn5 transposase to add an adapter to whole genomic DNA, and ii) RNA ligase to produce the transcriptome libraries in the same reaction. While this protocol is effective at preparing a whole genome and transcriptome library in a single tube, the protocol is characterized by a high degree of complexity.

Accordingly, there is a need for new schemes for the integration of DNA and RNA library preparation and target enrichment.

### SUMMARY

The present invention overcomes the aforementioned drawbacks by providing a system and method for total nucleic acid library preparation via template-switching as described by the following enumerated list:
1. A method, comprising:
   combining into a first reaction mixture:
      i) a nucleic acid sample including at least one double-stranded DNA, the double-stranded DNA having a first strand and a second strand, the second strand at least partially complementary to the first strand, each of the first strand and the second strand having a 5′ end and a 3′ end,
      ii) a first reverse transcriptase,
      iii) a first template-switch oligonucleotide excluding at least one nucleotide having a nucleobase selected from adenine, cytosine, guanine, and thymine, and
      iv) a first mixture of dNTPs excluding at least one dNTP selected from dATP, dCTP, dGTP, and dTTP, the at least one dNTP excluded from the mixture of dNTPs complementary to the at least one nucleotide excluded from the first template-switch oligonucleotide; and
   performing a first template-switching reaction with the first reaction mixture, comprising:
      adding at least one non-templated nucleotide to at least one of the 3′ ends of the double-stranded DNA with the reverse transcriptase, thereby forming a non-templated 3′ overhang on the double-stranded DNA;
      annealing the first template-switch oligonucleotide to the non-templated 3′ overhang of the double-stranded DNA; and
      extending the non-templated 3′ overhang of the double-stranded DNA with the reverse transcriptase, thereby forming a first nucleic acid product comprising the double-stranded DNA having at least one extended 3′ end complementary to the first template-switch oligonucleotide.
2. The method of item 1, wherein the nucleic acid sample further includes at least one RNA, the RNA having a 5′ end and a 3′ end.
3. The method of item 2, further comprising:
   combining into a second reaction mixture:
      i) the first nucleic acid product,
      ii) the RNA,
      iii) a second reverse transcriptase,
      iv) a second template-switch oligonucleotide, and
      v) a second mixture of dNTPs; and
   performing a second template-switching reaction with the second reaction mixture, comprising:
      synthesizing a polynucleotide complementary the RNA with the second reverse transcriptase, thereby forming a first primer extension product complementary to at least a portion of the RNA;
      adding at least one non-templated nucleotide to the 3′ end of the first primer extension product with the second reverse transcriptase, thereby forming a non-templated 3′ overhang on the first primer extension product;
      annealing the second template-switch oligonucleotide to the non-templated 3′ overhang of the first primer extension product; and
      extending the non-templated 3′ overhang of the first primer extension product with the second reverse transcriptase, thereby forming a second nucleic acid product comprising the first primer extension product having an extended 3′ end complementary to the second template-switch oligonucleotide.
4. The method of item 3, further comprising:
   combining into the second reaction mixture, a first oligonucleotide primer having a 3′ end complementary to the RNA; and
   wherein performing the second template-switching reaction with the second reaction mixture further comprises:
   annealing the 3′ end of the first oligonucleotide primer to the RNA; and
   extending the first oligonucleotide primer with the second reverse transcriptase, thereby forming the first primer extension product.
5. The method of item 3, wherein the nucleotide sequence of the first template-switch oligonucleotide differs from the nucleotide sequence of the second template switch oligonucleotide by at least one nucleotide.
6. The method of item 2, wherein the first template-switching reaction is incapable of forming a by-product comprising a complement of at least a portion of the RNA having a 3′ end complementary to the first template-switch oligonucleotide.
7. The method of item 1, further comprising terminating at least one of the 3′ ends of the first nucleic acid product.
8. The method of item 7, wherein the step of terminating comprises incorporating a dideoxynucleotide at the at least one 3′ end of the first nucleic acid product.
9. The method of item 1, wherein the first template-switch oligonucleotide includes at least one of a universal adapter sequence, a sample identified sequence, and a molecular identifier sequence.
10. The method of item 3, wherein the second template-switch oligonucleotide includes at least one of a universal adapter sequence, a sample identified sequence, and a molecular identifier sequence.
11. The method of item 3, wherein the method further comprises purifying the nucleic acid sample including the first nucleic acid product and the RNA prior to the step of combining into the second reaction mixture.
12. A method, comprising:
   combining into a first reaction mixture:
      i) a nucleic acid sample including at least one double-stranded DNA, the double-stranded DNA having a first strand and a second strand, the second strand at least partially complementary to the first strand, each of the first strand and the second strand having a 5′ end and a 3′ end,
      ii) a reverse transcriptase,
      iii) a first template-switch oligonucleotide having a 5′ domain and a 3′ domain, the 3′ domain of the first template-switch oligonucleotide excluding at least one nucleotide having a nucleobase selected from adenine, cytosine, guanine, and thymine,
      iv) a first mixture of dNTPs excluding at least one dNTP selected from dATP, dCTP, dGTP, and dTTP, the at least one dNTP excluded from the mixture of dNTPs complementary to the at least one nucleotide excluded from the 3′ domain of the first template-switch oligonucleotide, and
      v) a ddNTP complementary to the at least one nucleotide excluded from the 3′ domain of the first template-switch oligonucleotide; and
   performing a first template-switching reaction with the first reaction mixture, comprising:
      adding at least one non-templated nucleotide to at least one of the 3′ ends of the double-stranded DNA with the reverse transcriptase, thereby forming a non-templated 3′ overhang on the double-stranded DNA;
      annealing the first template-switch oligonucleotide to the non-templated 3′ overhang of the double-stranded DNA; and
      extending the non-templated 3′ overhang of the double-stranded DNA with the reverse transcriptase, thereby forming a first nucleic acid product comprising the double-stranded DNA having at least one extended 3′ end complementary to the 3′ domain of the first template-switch oligonucleotide.
13. The method of item 12, wherein the nucleic acid sample further comprises at least one RNA, the RNA having a 5′ end and a 3′ end.
14. The method of item 13, further comprising:
   combining into a second reaction mixture:
      i) the first nucleic acid product,
      ii) the RNA,
      iii) a second reverse transcriptase,
      iv) a second template-switch oligonucleotide, and
      v) a second mixture of dNTPs; and
   performing a second template-switching reaction with the second reaction mixture, comprising:
      synthesizing a polynucleotide complementary to the RNA with the second reverse transcriptase, thereby forming a first primer extension product complementary to at least a portion of the RNA;
      adding at least one non-templated nucleotide to the 3′ end of the first primer extension product with the second reverse transcriptase, thereby forming a non-templated 3′ overhang on the first primer extension product;
      annealing the second template-switch oligonucleotide to the non-templated 3′ overhang of the first primer extension product; and
      extending the non-templated 3′ overhang with the second reverse transcriptase, thereby forming a second nucleic acid product comprising the first primer extension product having an extended 3′ end complementary to the second template-switch oligonucleotide.
15. The method of item 14, further comprising:
   combining into the second reaction mixture, a first oligonucleotide primer having a 3′ end complementary to the RNA; and
   wherein performing the second template-switching reaction with the second reaction mixture further comprises:
   annealing the 3′ end of the first oligonucleotide primer to the RNA; and
   extending the first oligonucleotide primer with the second reverse transcriptase, thereby forming the first primer extension product.
16. The method of item 14, wherein the nucleotide sequence of the first template-switch oligonucleotide differs from the nucleotide sequence of the second template switch oligonucleotide by at least one nucleotide.
17. The method of item 13, wherein the first template-switching reaction is incapable of forming a by-product comprising a complement of at least a portion of the RNA having a 3′ end complementary to the first template-switch oligonucleotide.
18. The method of item 12, wherein the 5′ domain of the first template-switch oligonucleotide includes the at least one nucleotide excluded from 3′ domain of the first template-switch oligonucleotide, and wherein the first nucleic acid product includes at least one 3′ end terminating in the ddNTP.
19. The method of item 12, wherein the 3′ domain of the first template-switch oligonucleotide includes at least one of a universal adapter sequence, a sample identified sequence, and a molecular identifier sequence.
20. The method of item 14, wherein the second template-switch oligonucleotide includes at least one of a universal adapter sequence, a sample identified sequence, and a molecular identifier sequence.
21 The method of item 14, wherein the method further comprises purifying the nucleic acid sample including the first nucleic acid product and the RNA prior to the step of combing into the second reaction mixture.
22. The method of item 3 or 14, wherein the second nucleic acid product includes a second target sequence, and wherein the method further comprises:
   amplifying at least a portion of the second nucleic acid product in a second amplification reaction mixture including:
      i) the second nucleic acid product,
      ii) a first primer having a 3′ end corresponding to at least a 5′ end of the second template-switch oligonucleotide, and
      iii) a second primer having a 3′ end corresponding to the second target sequence.
23. The method of any one of the preceding items, wherein the first nucleic acid product includes a first target sequence, and wherein the method further comprises:
   amplifying at least a portion of the first nucleic acid product in a first amplification reaction mixture including:
      i) the first nucleic acid product,
      ii) a first primer having a 3′ end corresponding to at least a 5′ end of the first template-switch oligonucleotide, and
      iii) a second primer having a 3′ end corresponding to the first target sequence.
24. The method of any one of the preceding items, wherein each of the 3′ ends of the first nucleic acid product comprises an extended 3′ end complementary to the first template-switch oligonucleotide.
25. The method of any one of the preceding items wherein the second template-switching reaction is incapable of forming a by-product comprising the first nucleic acid product having a 3′ end complementary to the second template-switch oligonucleotide.
26. The method of any one of the preceding items wherein the nucleic acid sample includes a plurality of DNA.
27. The method of any one of the preceding items wherein the nucleic acid sample includes a plurality of RNA.
28. The method of any one of the preceding items wherein the reverse transcriptase is selected from a Moloney Murine Leukemia Virus (MMLV) reverse transcriptase, an Avian Myeloblastosis Virus (AMV) reverse transcriptase, and a mutant thereof.
29. The method of item 4 or 15 wherein the 3′ end of the first oligonucleotide primer is a poly-dT sequence.
30. The method of item 4 or 15, wherein the 3′ end of the first oligonucleotide primer is a target specific sequence.

31. The method of item 4 or 15, wherein the 3′ end of the first oligonucleotide primer is a random sequence. 32. The method of any one of the preceding items, wherein the first template-switch oligonucleotide comprises a stuffer region.
33. The method of item 4 or 15, wherein the second template-switch oligonucleotide comprises a stuffer region.
34. The method of item 4 or 15, wherein the 3′ end of the first oligonucleotide primer is complementary to an exonic region of the RNA.
35. The method of item 7, wherein the step of terminating comprises incorporating a dideoxynucleotide at the at least one 3′ end of the first nucleic acid product with a terminal transferase.
36. The method of item 35, wherein the terminal transferase is Taq DNA polymerase.
37. The method of any one of the preceding items, wherein performing the first template-switching reaction with the first reaction mixture further comprises adding at least three non-templated nucleotides to at least one of the 3′ ends of the double-stranded DNA with the reverse transcriptase.
38. The method of item 3 or 14, wherein performing the second template-switching reaction with the second reaction mixture comprises adding at least three non-templated nucleotides to the 3′ end of the first primer extension product with the reverse transcriptase.
39 The method of item 2 or 13, further comprising recovering the nucleic acid sample including the first nucleic acid product and the at least one RNA as a second nucleic acid sample.
40. A method, comprising:
   combining into a first reaction mixture:
      i) a nucleic acid sample including at least one double-stranded DNA, the double-stranded DNA having a first strand and a second strand, the second strand at least partially complementary to the first strand, each of the first strand and the second strand having a 5′ end and a 3′ end, and at least one RNA, the RNA having a first strand having a 5′ end and a 3′ end,
      ii) a reverse transcriptase,
      iii) a first template-switch oligonucleotide excluding at least one nucleotide having a nucleobase selected from adenine, cytosine, guanine, and thymine, and
      iv) a first mixture of dNTPs excluding at least one dNTP selected from dATP, dCTP, dGTP, and dTTP, the at least one dNTP excluded from the mixture of dNTPs complementary to the at least one nucleotide excluded from the first template-switch oligonucleotide; and
   performing a first template-switching reaction with the first reaction mixture, comprising:
      adding at least one non-templated nucleotide to at least one of the 3′ ends of the double-stranded DNA with the reverse transcriptase, thereby forming a non-templated 3′ overhang on the double-stranded DNA;
      annealing the first template-switch oligonucleotide to the non-templated 3′ overhang of the double-stranded DNA; and
      extending the non-templated 3′ overhang of the double-stranded DNA with the reverse transcriptase, thereby forming a first nucleic acid product comprising the double-stranded DNA having at least one extended 3′ end complementary to the first template-switch oligonucleotide.
41. A method for carrying out a template-switching reaction on a nucleic acid sample including at least one double-stranded DNA and at least one RNA, the method comprising:
   performing a first template-switching reaction on the nucleic acid sample in the absence of at least one dNTP selected from dATP, dCTP, dGTP, and dTTP, thereby forming a first nucleic acid product comprising the double-stranded DNA having at least one extended 3′ end complementary to a first template-switch oligonucleotide; and
   performing a second template-switching reaction on the nucleic acid sample, thereby forming a second nucleic acid product comprising a first primer extension product complementary to at least a portion of the RNA, the first primer extension product having an extended 3′ end complementary to the second template-switch oligonucleotide.
42. A kit for performing a template-switching reaction on a nucleic acid sample including at least one double-stranded DNA and at least one RNA, the kit comprising:
   a first template-switch oligonucleotide excluding at least one nucleotide having a nucleobase selected from adenine, cytosine, guanine, and thymine; and
   a first mixture of dNTPs excluding at least one dNTP selected from dATP, dCTP, dGTP, and dTTP, the at least one dNTP excluded from the mixture of dNTPs complementary to the at least one nucleotide excluded from the first template-switch oligonucleotide.
43. The kit of item 42, further comprising:
   a second template-switch oligonucleotide; and
   a second mixture of dNTPs.
44. A kit for performing a template-switching reaction on a nucleic acid sample including at least one double-stranded DNA and at least one RNA, the kit comprising:
   a first template-switch oligonucleotide having a 5′ domain and a 3′ domain, the 3′ domain of the first template-switch oligonucleotide excluding at least one nucleotide having a nucleobase selected from adenine, cytosine, guanine, and thymine;
   a first mixture of dNTPs excluding at least one dNTP selected from dATP, dCTP, dGTP, and dTTP, the at least one dNTP excluded from the mixture of dNTPs complementary to the at least one nucleotide excluded from the 3′ domain of the first template-switch oligonucleotide; and
   a ddNTP complementary to the at least one nucleotide excluded from the 3′ domain of the first template-switch oligonucleotide.
45. The kit of item 44, further comprising:
   a second template-switch oligonucleotide; and
   a second mixture of dNTPs.
46. The method of item 3 or 14 wherein at least one of the first template-switch oligonucleotide and the second template-switch oligonucleotide comprises ribonucleotides.
47. The method of item 46, further comprising contacting at least one of the first template-switch oligonucleotide and the second template-switch oligonucleotide with a ribonuclease.
48. The method of items 3 or 14, wherein at least one of the first template-switch oligonucleotide and the second template-switch oligonucleotide comprises a 5′ modification selected from a nucleotide analog, a linkage modification, a terminal modification, and a fluorescent label.
49. The method of item 1 or 3, wherein 3′ end of at least one of the first template-switch oligonucleotide and the second template-switch oligonucleotide comprises a homopolymer sequence of at least three nucleotides.
50. The method of item 49, wherein the homopolymer sequence is selected from polyriboguanosine, polyguanosine, polyribocytidine, and polycytidine.
51. The method of item 12, wherein at least one of the first template-switch oligonucleotide and the second template-switch oligonucleotide comprises at least one 2′-O-Methyl nucleoside modification.
52. The method of items 1 or 12, wherein the first template-switch oligonucleotide further excludes uracil.
53. The method of items 1 or 12, wherein the first mixture of dNTPs further excludes dUTP.
54. The method of item 12, wherein the 5′ domain of the first template-switch oligonucleotide comprises the at least one nucleotide excluded from the 3′ domain of the first template-switch oligonucleotide.
55. The method of item 12 or 54, wherein the ddNTP further comprises a capture moiety.
56. The method of item 55, wherein the capture moiety is selected from biotin and desthiobiotin.

57. The method of item 1 or 12, wherein the first mixture of dNTPs includes at least one dNTP having a capture moiety.
58. The method of item 57, wherein the capture moiety is selected from biotin and desthiobiotin.
59. The method of item 3 or 14, wherein the second mixture of dNTPs includes at least one capture moiety.
60. The method of item 59, wherein the capture moiety is selected from biotin and desthiobiotin.
61. The method of item 4 or 15, wherein the first oligonucleotide primer includes at least one capture moiety.
62. The method of item 61, wherein the capture moiety is selected from biotin and desthiobiotin.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic illustration of a method for preparing a total nucleic acid sample according to the present disclosure.

FIG. 2A is a schematic diagram depicting the components of a total nucleic acid sample according to the present disclosure.

FIG. 2B is a schematic diagram depicting the components of the total nucleic acid sample of FIG. 2A following the addition of a non-templated 3′ overhang to the 3′ ends of a double-stranded DNA.

FIG. 2C is a schematic diagram depicting the components of the total nucleic acid sample of FIG. 2B following annealing of a first template-switch oligonucleotide to the non-templated 3′ overhang.

FIG. 2D is a schematic diagram depicting the components of the total nucleic acid sample of FIG. 2C following extension of the non-templated 3′ overhang of the double-stranded DNA, thereby forming a first nucleic acid product comprising the double-stranded DNA having at least one extended 3′ end complementary to the first template-switch oligonucleotide.

FIG. 3A is a schematic diagram depicting the first nucleic acid product and the RNA of FIG. 2D combined with a second template-switch oligonucleotide and a primer.

FIG. 3B is a schematic diagram depicting the components of FIG. 3A following the synthesis of a polynucleotide complementary the RNA, thereby forming a first primer extension product, and addition of a non-templated 3′ overhang to the 3′ end of the first primer extension product.

FIG. 3C is a schematic diagram depicting the components of FIG. 3B following annealing of a second template-switch oligonucleotide to the non-templated 3′ overhang.

FIG. 3D is a schematic diagram depicting the components of FIG. 2C following extension of the non-templated 3′ overhang of the first primer extension product., thereby forming a second nucleic acid product comprising the first primer extension product having an extended 3′ end complementary to the second template-switch oligonucleotide.

FIG. 4A is a schematic diagram depicting the components of FIG. 3D combined with primer pairs for amplification.

FIG. 4B is a schematic diagram depicting the product of the amplification reaction illustrated in FIG. 4A.

Like numbers will be used to describe like parts from Figure to Figure throughout the following detailed description.

### DETAILED DESCRIPTION

### I. Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Approximately: As used herein, the term “approximately” or “about”, as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level, and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises or consists of an organism, such as an animal or human. In some embodiments, a biological sample is comprises or consists of biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom, etc. In some embodiments, a biological sample is comprises or consists of cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Comprising: A composition or method described herein as “comprising” one or more named elements or steps is open-ended, meaning that the named elements or steps are essential, but other elements or steps may be added within the scope of the composition or method. It is to be understood that composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Designed: As used herein, the term “designed” refers to an agent (i) whose structure is or was selected by the hand of man; (ii) that is produced by a process requiring the hand of man; and/or (iii) that is distinct from natural substances and other known agents.

Determine: Those of ordinary skill in the art, reading the present specification, will appreciate that “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Sample: As used herein, the term “sample” refers to a substance that is or contains a composition of interest for qualitative and or quantitative assessment. In some embodiments, a sample is a biological sample (i.e., comes from a living thing (e.g., cell or organism). In some embodiments, a sample is from a geological, aquatic, astronomical, or agricultural source. In some embodiments, a source of interest comprises or consists of an organism, such as an animal or human. In some embodiments, a sample for forensic analysis is or comprises biological tissue, biological fluid, organic or non-organic matter such as, e.g., clothing, dirt, plastic, water. In some embodiments, an agricultural sample, comprises or consists of organic matter such as leaves, petals, bark, wood, seeds, plants, fruit, etc.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: As used herein, the word “synthetic” means produced by the hand of man, and therefore in a form that does not exist in nature, either because it has a structure that does not exist in nature, or because it is either associated with one or more other components, with which it is not associated in nature, or not associated with one or more other components with which it is associated in nature.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, a variant may also have one or more functional defects and/or may otherwise be considered a “mutant”. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### II. Detailed Description of Certain Embodiments

As also discussed above, in various situations it may be useful to provide integrated DNA and RNA library preparation and target enrichment schemes. In one aspect, simultaneous generation of RNA and DNA sequencing libraries enables the collection of both DNA and RNA sequencing data from the same sample using next-generation sequencing (NGS). RNA sequencing data provides verification of DNA variant calls and can assist the identification of driver mutations by quantifying expressed transcripts, allele-specific expression, and RNA editing. However, generation of paired DNA and RNA sequencing libraries for NGS from the same biological specimen is not without challenges. Library construction from DNA and RNA from a single sample is typically achieved by purifying total nucleic acid (TNA), which is then split into two different samples and treated with either DNase I to recover the RNA, or RNase A, to recover the DNA. This approach results in the loss of half the RNA and half the DNA. Moreover, the addition of DNase I or RNase A can result in degradation of the desired TNA fraction, which is problematic when only a small amount of sample may be available to work with. Other existing commercial products allow for the sequential isolation of RNA and DNA fractions in a single protocol; however once the DNA and RNA is isolated, the sample are processed separately, necessitating additional time and effort.

Accordingly, there is a need for a system and method to generate RNA and DNA sequencing libraries from a single sample originating with TNA. In this approach, the DNA and RNA portions are never physically separated and instead are prepared for sequencing in a single tube. In another aspect, there is a need for schemes that are i) compatible with automation platforms (e.g., liquid handling robots), ii) accommodating of high or low-quality TNA samples, and iii) capable of discriminating between reads originating from either DNA and RNA following sequencing.

The present disclosure provides methods and kits for the efficient addition of unique adapters to RNA, DNA or both, where DNA and RNA are present in a single sample, and never separated. The disclosed approach further allows for the selection and enrichment of DNA, RNA or both, for example, through the use of amplification and sequencing. Moreover, sequencing reads that originated from either the DNA or the RNA strand are readily differentiated with high confidence using the disclosed system and method. It is anticipated that the methods disclosed herein provide for a simpler workflow having fewer steps compared with existing workflows. Finally, it is anticipated that the disclosed methods are compatible with a variety of nucleic acid sample types including both fragmented and high quality TNA.

In one aspect, the present disclosure provides a method for integrated TNA library preparation based on the terminal transferase activity and template-switching ability of reverse transcriptase (RT) enzymes such as the MMLV RT. The use of an RT enzyme is an effective way to add a known sequence or adapter to the end of a full cDNA sequence. The mechanism involves the ability of the RT to add non-templated nucleotides to the 3′ end of a complementary DNA (cDNA) strand. Once the end of the template (which is normally the 5′ end of an RNA molecule) is reached, the terminal transferase activity of the RT enzyme catalyzes the addition of non-templated nucleotides to the 3′ end of the growing cDNA strand. The resulting 3′ overhang facilitates the annealing of a complementary 3′ oligo, referred to herein as a template-switching oligo (TSO). The 3′ non-template overhang is typically a poly-cytosine (e.g., CCC) with the complementary TSO including a 3′ polyriboguanosine (e.g., rGrGrG, where ‘r’ represents a ribonucleotide base); however, it will be appreciated that a terminal transferase can generate alternative 3′ overhangs depending on the makeup of the dNTP pool and the specificity of the enzyme. With the TSO annealed to the non-template overhang, the RT enzyme subsequently switches templates, moving from the initially reverse-transcribed RNA template to the new TSO template. The end result is the attachment of a 3′ new sequence to the 3′ cDNA that is the reverse complement of the TSO. Example template-switching applications are described for example, in U.S. Pat. No. 5,962,271 to Chenchik et al., the entirety of which is incorporated herein by reference.

It is known that the template-switching mechanism of RT is compatible with both DNA and RNA templates; however, no existing integrated library preparation schemes make use of the template-switching mechanism of RT for the preparation of both DNA and RNA present in the same sample. One challenge to achieving the use of the template-switching mechanism of RT for integrated library preparation relates to controlling for the selective addition of different TSO derived sequences to RNA and DNA when both nucleic acids are present in the same sample. Nonetheless, the present disclosure provides a system and method for TNA library preparation via template-switching.

In general, the present disclosure is based on the surprising discovery that a library preparation scheme involving a reverse transcriptase enzyme possessing template-switching activity can be applied to the preparation of a TNA sample (i.e., a sample including both DNA and RNA). Advantageously, the library preparation scheme is a one-pot approach capable of selectively and distinctly tagging both DNA and RNA present in the same sample. Selective and distinguishable tagging is achieved through two distinct template-switching reactions performed sequentially on a TNA sample. Each template-switching reaction is selective for the preparation of either dsDNA or RNA even though both the dsDNA and RNA are present in the same reaction. The resulting product of the scheme includes a DNA-derived nucleic acid product having a first adapter sequence and an RNA-derived nucleic acid product having a second adapter sequence that is different from the first adapter sequence. Accordingly, following sequencing of the nucleic acid products, the resulting reads are readily associated with either the original RNA template(s) or the original DNA template(s) present in the TNA sample.

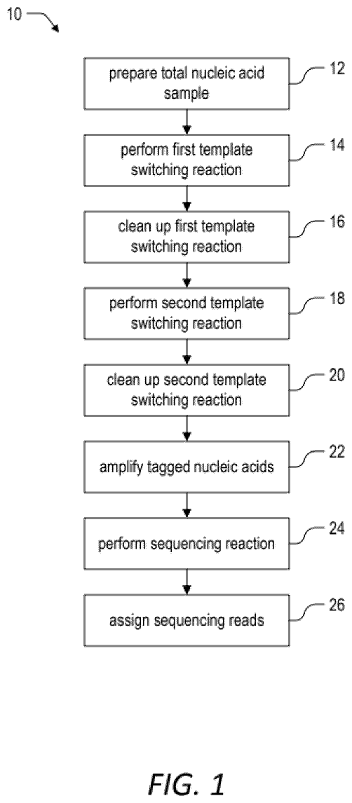

Turning now to FIG. 1, an embodiment of a method 10 according to the present disclosure includes a step 12 of preparing a TNA sample. In one aspect, the TNA sample includes all nucleic acids extracted and isolated from a biological sample. A TNA sample can include genomic DNA, messenger RNA (mRNA), ribosomal RNA (rRNA), and the like. In one aspect, the step 12 can include preparing fragmented, blunt-ended DNA. As will be appreciated by one of ordinary skill in the art, blunt dsDNA can be prepared in various ways. DNA, including genomic DNA can be fragmented using any suitable approach, including enzymatic fragmentation, mechanical sheering, sonication, or the like. DNA fragments can be blunt ended using any suitable approach including both fill-in reactions (e.g., via polymerase) and chew-back reactions (e.g., via exonuclease). In another aspect, it can be desirable to enrich for a portion of the total RNA present in the TNAs. One enrichment method includes rRNA reduction, for example, using RNaseH. Yet other preparation steps can be applied to the TNA sample in the step 12 as will be appreciated by one of ordinary skill in the art.

Following preparation in the step 12, the TNA sample includes both blunt dsDNA and RNA. The TNA sample prepared in the step 12 is then subjected to two separate template-switching reactions performed sequentially. A step 14 of the method 10 includes performing a first template-switching reaction. In the first template-switching reaction, reaction conditions are provided so that the template-switching reaction occurs only on the blunt dsDNA portion of the TNA. In one embodiment, the first template-switching reaction mixture includes the nucleic acid sample, a first reverse transcriptase, a first TSO (TSO-A) excluding at least one nucleotide having a nucleobase selected from adenine, cytosine, guanine, and thymine, and, a first mixture of dNTPs excluding at least one dNTP selected from dATP, dCTP, dGTP, and dTTP. The at least one dNTP excluded from the mixture of dNTPs is complementary to the at least one nucleotide excluded from the first template-switch oligonucleotide. For example, if TSO-A excludes only nucleotides having the nucleobase thymine, then the first mixture of dNTPs would exclude the complementary dATP. Omitting at least one type of nucleobase from the first TSO (i.e., TSO-A) and excluding the complementary dNTP from the first mixture of dNTPs in the reaction ensures that only the dsDNA and not the RNA undergoes template-switching. In particular, this design eliminates non-specific priming and extension associated with RNA templates present in the reaction, as replication of RNA templates is arrested when the RT encounters a base complementary to the missing dNTP. The lack of all four canonical dNTPs thus prevents the RT from completely extending to the 5′ terminus of the RNA and from catalyzing the template-switching reaction associated with the RNA present in the TNA sample. Notably, the inventors have observed that the template-switching reaction appears to be highly efficient on the dsDNA present in the sample with the majority (i.e., >50%) of blunt dsDNA undergoing template-switching. More notably, other schemes can be implemented to achieve template-switching on only dsDNA templates. For example, dideoxynucleotide triphosphates (ddNTPs) can be used as described below.

It will be appreciated that, in general, the nucleobase uracil and thymine can be used interchangeably. For example, when the first TSO excluding the nucleobase adenine, then the first mixture of dNTPs excludes dTTP, which is complementary to adenine. In this case, it may be useful to further exclude dUTP from the first mixture of dNTPs. Similarly, when the first TSO excludes thymine, it may be useful to further exclude the nucleobase uracil from the first TSO. Accordingly, in one aspect of the present disclosure, when the first template-switch oligonucleotide excludes thymine, the first template-switch oligonucleotide further excludes uracil. In another aspect of the present disclosure, when the first mixture of dNTPs excludes dTTP, the first mixture of dNTPs further excludes dUTP With continued reference to the step 14 of the method 10 in FIG. 1, the composition resulting from the first reaction mixture includes a first nucleic acid product including the dsDNA having at least one extended 3′ end complementary to the first TSO. The composition also includes the unprocessed RNA, TSO-A, the RT enzyme, and any remaining reagents, such as dNTPs. In preparation for the second template-switching reaction of the disclosed method, the method 10 can include a step 16 in which the reaction mixture is subjected to a clean-up step to recover the first nucleic acid product and the RNA away from the other components in the composition resulting from the first reaction mixture. The step 16 can include any suitable clean-up scheme to recover the nucleic acids away from the other components of the reaction. Example clean-up schemes include column and bead-based nucleic acid recovery methods such as solid-phase reversible immobilization (SPRI) on carboxylated paramagnetic beads, solvent (e.g., ethanol) based extraction protocols, the like, and combinations thereof.

A next step 18 of the method 10 includes performing a second template-switching reaction. The setup of the second template-switching reaction is designed to achieve template-switching on the remaining TNA that did not undergo the template-switching reaction in the first template-switching reaction step (i.e., the RNA in the present example). The second template-switching reaction includes the first nucleic acid product, the RNA, a reverse transcriptase having template-switching activity, a second template-switch oligonucleotide (TSO-B), and a second mixture of dNTPs. The TSO-B has a sequence that is distinguishable from the sequence of TSO-A in order to differentiate between the nucleic acid product derived from the dsDNA template(s) and the nucleic acid products derived from the RNA template(s). In one aspect, the nucleotide sequence of the first template-switch oligonucleotide differs from the nucleotide sequence of the second template-switch oligonucleotide by at least one nucleotide. In another aspect, the nucleotide sequence of the first template-switch oligonucleotide differs from the nucleotide sequence of the second template-switch oligonucleotide by at least 2, 3, 4, 5, 6, 7, 8, 9, 10, or more nucleotides.

In another aspect, the second mixture of DNA includes all four canonical dNTPs, such that the reverse transcriptase is able to completely replicate the RNA template in order to enable the template-switching reaction to take place at the 5′ end of the RNA template. Notably, the second template-switching reaction can optionally include at least one primer designed to enable priming of replication of the RNA template with the reverse transcriptase. The primer can be an oligo dT primer, a target specific primer, a randomer, or a combination thereof.

With continued reference to the step 18 of the method 10 in FIG. 1, the second template-switching reaction facilitates priming off of the RNA template(s), reverse transcription for cDNA synthesis and template-switching using TSO-B. It should be appreciated that any unreacted blunt dsDNA that did not undergo template-switching in the first reaction can undergo template-switching in the second reaction. Accordingly, if it is desirable to prevent the addition of sequence complementary to the second TSO (i.e., TSO-B) to the dsDNA, then steps should be taken to ensure that complete conversion of the dsDNA to the first nucleic acid product occurs in the first template-switching reaction. Alternatively, or in addition, steps can be taken to eliminate unreacted dsDNA from the second reaction. This and other approaches will be described in further detail herein.

The method 10 further includes a step 20 of cleaning up the second template-switching reaction. The composition resulting from the second reaction mixture includes the first nucleic acid product, a second nucleic acid product including the cDNA derived from the RNA template(s). The cDNA has an extended 3′ end complementary to TSO-B. The composition also includes the unprocessed RNA, TSO-B, the RT enzyme, and any remaining reagents, such as dNTPs. In preparation for downstream processing (if any) ahead of sequencing, the composition resulting from the second reaction mixture can be subjected to a clean-up step to recover the first and second nucleic acid products away from the other components in the composition resulting from the second reaction mixture.

The method 10 further includes a step 22 of amplifying the TSO sequence tagged nucleic acids. In one aspect, the first and second nucleic acid products can be amplified by PCR using various methods. The amplification step 22 can further include attachment of adapters compatible with a selected sequencing platform. The amplification reaction can be designed to amplify the DNA-derived product, the RNA derived product, or both by including a primer specific for either or both of the TSO-A an TSO-B sequences. The TSO-specific primers can further be paired with target-specific primers to enable enrichment of specific nucleic acid sequences. It will be appreciated that the two-step template-switching approach and subsequent amplification methods disclosed herein are amenable to a variety of modifications in order to accommodate different desired outcomes as will become apparent from present disclosure.

Following the first and second template-switching reactions in the step 14 and the step 18 respectively, a step 24 of the method 10 includes performing a sequencing reaction of the product nucleic acids. In one aspect, the products of the template-switching reactions are sequenced directly without amplification. In another aspect, the products resulting from amplification in the step 22 are sequenced. Any suitable sequencing platform can be used, including short read and long read platforms, sequencing by synthesis platforms, and nanopore-based sequencing platforms. The method 10 further includes a step 26 of assigning sequencing reads. Based on the TSO sequence detected, a given sequencing read can precisely and accurately assigned as having resulted from either a DNA or an RNA molecule originally present in the TNA sample.

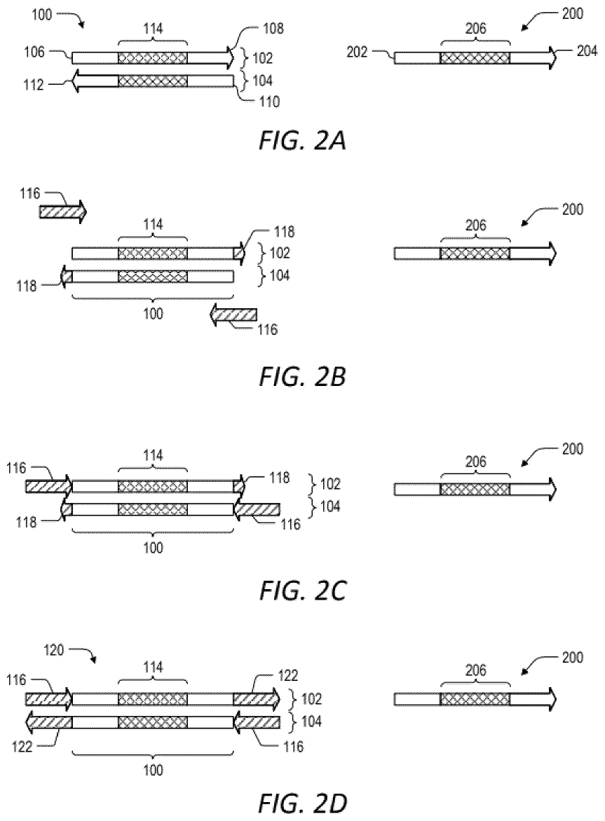

Turning now to FIG. 2A, a TNA sample can include at least one double-stranded DNA 100 and at least one RNA 200. The double-stranded DNA 100 has a first strand 102 and a second strand 104. The second strand 104 is at least partially complementary to the first strand 102. Moreover, the first strand 102 has a 5′ end 106 and a 3′ end 108, and the second strand 104 has a 5′ end 110 and a 3′ end 112. Notably, the directionality of the first strand 102 and the second strand 104, along with all other illustrated nucleic acids, is indicated by the use of arrowheads throughout the Figures. The double-stranded DNA 100 further defines a first target sequence 114.

With reference to FIG. 2B, the double stranded DNA 100 is combined into a first reaction mixture with a first reverse transcriptase (not shown), and a first template-switch oligonucleotide or TSO 116 and a first mixture of dNTPs (not shown). The first TSO 116 excludes at least one nucleotide having a nucleobase selected from adenine, cytosine, guanine, and thymine. The first mixture of dNTPs excludes at least one dNTP selected from dATP, dCTP, dGTP, and dTTP, where the at least one dNTP excluded from the mixture of dNTPs is complementary to the at least one nucleotide excluded from the first TSO 116.

The first reaction mixture includes the necessary components for performing a first template-switching reaction with the first reaction mixture. In one aspect, the first template-switching reaction includes adding at least one non-templated nucleotide to at least one of the 3′ ends of the double-stranded DNA 100 with the reverse transcriptase, thereby forming a non-templated 3′ overhang 118 on the double-stranded DNA 100. The non-templated 3′ overhang 118 is complementary to the a 5′ end of the TSO 116, allowing for annealing of the first TSO 116 to the non-templated 3′ overhang 118 of the double-stranded DNA 100 (FIG. 2C).

Following annealing of the TSO 116, the non-templated 3′ overhang 118 of the double-stranded DNA 100 is extended with the reverse transcriptase, thereby forming a first nucleic acid product 120 comprising the double-stranded DNA 100 having at least one extended 3′ end 122 complementary to the first template-switch oligonucleotide 116.

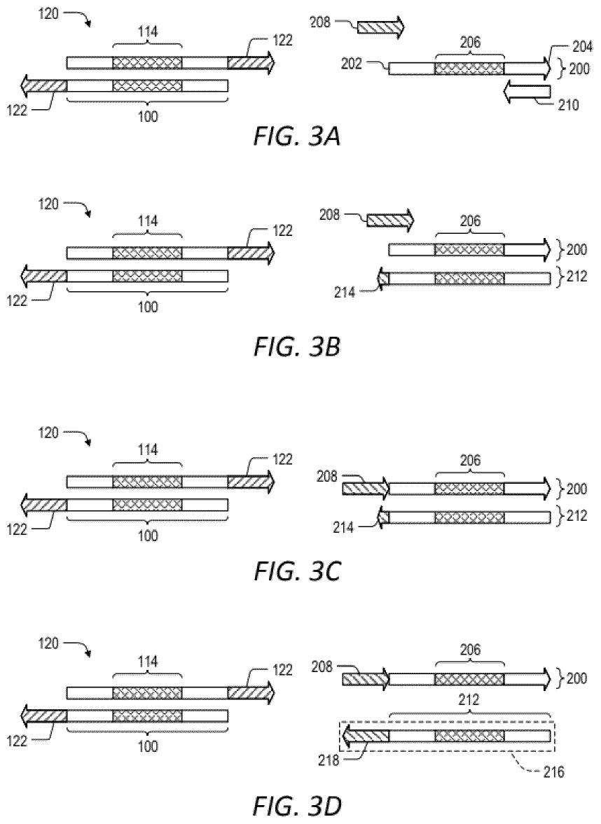

Turning now to FIG. 3A, a second template-switching reaction can be carried out on the at least one RNA 200. The RNA 200 has a 5′ end 202 and a 3′ end 204, and further defines a second target sequence 206. The RNA 200 is combined into a second reaction mixture including the first nucleic acid product 120, a second reverse transcriptase (not shown), a second template-switch oligonucleotide 208, and a second mixture of dNTPs (not shown). In one aspect, the nucleotide sequence of the first template-switch oligonucleotide 116 differs from the nucleotide sequence of the second template switch oligonucleotide 208 by at least one nucleotide. The second reaction mixture can optionally include a first oligonucleotide primer 210 that is at least partially complementary to the RNA 200.

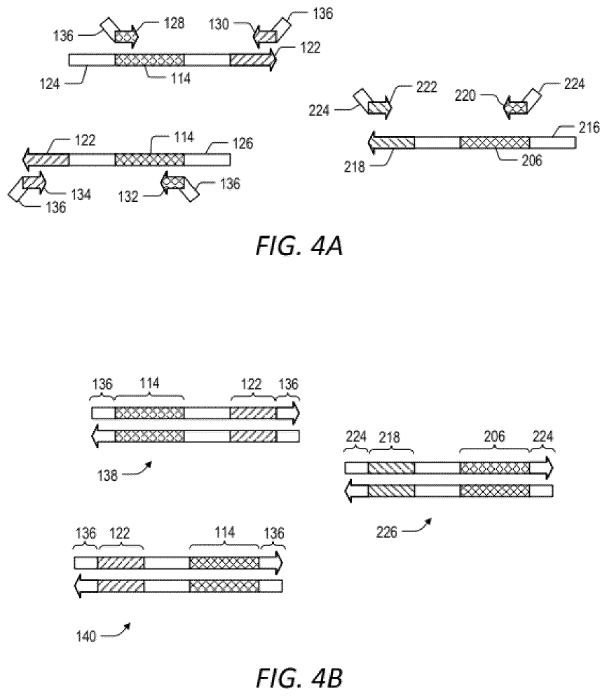

Referring to FIGS. 3B and 3C, performing a second template-switching reaction with the second reaction mixture includes synthesizing a polynucleotide complementary the RNA 200 with the second reverse transcriptase, thereby forming a first primer extension product 212 that is complementary to at least a portion of the RNA 200. In one aspect, performing the second template-switching reaction with the second reaction mixture further includes annealing the 3′ end of the first oligonucleotide primer 210 to the RNA 200 and extending the first oligonucleotide primer with the second reverse transcriptase, thereby forming the first primer extension product 212. Moreover, at least one non-templated nucleotide can be added to the 3′ end of the first primer extension product 212 with the second reverse transcriptase, thereby forming a non-templated 3′ overhang 214 on the first primer extension product 212. The second template-switch oligonucleotide 208 can anneal to the non-templated 3′ overhang 214 of the first primer extension product 212, and the non-templated 3′ overhang 214 of the first primer extension product 212 can be extended with the second reverse transcriptase, thereby forming a second nucleic acid product 216 comprising the first primer extension product 212 having an extended 3′ end 218 complementary to the second template-switch oligonucleotide (FIG. 3D) Turning to FIG. 4, the first nucleic acid product 120 and the second nucleic acid product 216 can be amplified using various methods to attach sequencing adapters to each end. In one aspect, a first nucleic acid product 120 includes a top strand 124 and a bottom strand 126. Each of the top strand 124 and the bottom strand 126 can be selectively amplified using distinct primer pairs. For example, a first primer pair for amplifying the top strand 124 can include a target specific primer 128 and a primer 130 specific to the extended 3′ end 122, while a second primer pair for amplifying the bottom strand 126 can include a target specific primer 132 and a primer 134 specific to the extended 3′ end 122. In another aspect, the second nucleic acid product 216 can be selectively amplified using distinct primer pairs different from those used to amplify the first nucleic acid product 120. For example, a first primer pair for amplifying the second nucleic acid product 216 can include a target specific primer 220 and a primer 222 specific to the extended 3′ end 218.

Notably, each of the primers 128, 130, 132, and 134 can include a 5′ tail 136 defining an adapter sequence. The 5′ tails 136 can have define the same or different sequences and can include sequencing platform specific sequences, sample identifier sequences, molecular identifier sequences, the like and combinations thereof. Similarly, each of the primers 220 and 222 can include a 5′ tail 224 defining an adapter sequence. The 5′ tails 224 can have define the same or different sequences and can include sequencing platform specific sequences, sample identifier sequences, molecular identifier sequences, the like and combinations thereof.

Turning to FIG. 4B, the products of the amplification step shown in FIG. 4A are uniquely identified by a corresponding TSO sequence, and are further compatible for sequencing on a selected platform due to the addition of adapter sequences. In a first example, a first product 138 is derived from the top strand 124 following amplification with the primer 128 and the primer 130. The first product 138 includes the first target sequence 114, the extended 3′ end 122 including a TSO-derived sequence and common sequences corresponding to the 5′ tails 136. In a second example, a second product 140 is derived from the bottom strand 126 following amplification with the primer 132 and the primer 134. The second product 140 includes the first target sequence 114, the extended 3′ end 122 including a TSO-derived sequence and common sequences corresponding to the 5′ tails 136. In a third example, a third product 226 is derived from second nucleic acid product 216 following amplification with the primer 220 and the primer 222. The third product 226 includes the second target sequence 206, the extended 3′ end 218 including a TSO-derived sequence and common sequences corresponding to the 5′ tails 224.

It will be appreciated that various modifications can be made to the disclosed methods in order to improve the template-switching reaction on DNA or ensure only the DNA undergoes template-switching in the first template-switching reaction. In one aspect, The first TSO or the second TSO-B can include one or more i) nucleotide analogs such as locked nucleic acids (LNA), fluoro-beta-D-arabinonucleic acid (FANA), 2′-O-Methyl RNA, 2′-fluoro RNA, ii) linkage modifications such as phosphorothioates, 3′-3′ and 5′-5′ reversed linkages, iii) 5′ end modifications, 3′ end modifications or a combination thereof, such as amino, biotin, Digoxigenin11dUTP, phosphate, thiol, dye, and quencher modifications, iv) one or more fluorescently labeled nucleotides, or v) any other feature that provides a desired functionality to the template switch oligonucleotide.

In another aspect, a unique sequence can be included in the first TSO that would clearly identify the reads originating from the first template-switching reaction and thus indicate which products or sequencing reads were derived from the dsDNA portion of the TNA.

In another aspect, buffer conditions can be optimized so that template switching on the DNA is preferred rather than on the RNA.

In another aspect, template-switching reactions can be improved through the use of TSO having a 3′ terminal sequence selected from NNN and rNrNrN (where r indicates an RNA base and N indicates a nucleic acid base). In one example, the 3′ terminal sequence of the TSO is a homopolymer (e.g., AAA, rArArA, CCC, rCrCrC, TTT, rTrTrT, GGG, or rGrGrG). In another example, the 3′ terminal sequence of the TSO is a heteropolymer (e.g., CGC, rCrGrC, or the like). In yet another example, a composition is prepared having a plurality of TSO with different 3′ terminal sequences. For example, a TSO composition can include equal portions of TSO having two different 3′ terminal sequences.

In another aspect, the dNTP excluded from the mixture of dNTPs is selected to enhance template switching.

In another aspect, a TSO can include a unique molecular identifier (UMI)—also known as a unique molecular identifier (UID) or barcode sequence. The UMI can have a variable length and sequence. TSO can include a UMI at the 5′ end, the 3′ end, or at an intermediate location. In one aspect, the UMI is 2, 3, 4, 5, 6, 7, 8, 9, 10, or more nucleotides in length.

In another aspect, a template-switching enhancer region can be positioned adjacent to the 5′ -region of the 3′ -end of the TSO. An enhancer region includes the first bases to be incorporated following successful template switching. The enhancer region sequence can be selected to enhance the template-switching reaction.

In another aspect, the disclosed method can include the use of a mixture of at least two different first or second TSO, where the different TSO include a variable stuffer region located 5′ of the 3′ terminal sequence. The stuffer region includes a nucleotide sequence selected to alleviate a loss in complexity that can result when reading through the sequence derived from the 3′ terminus of the TSO (as this sequence may be identical for all TSO used in the template-switching reaction). As will be appreciated, certain sequencing instruments benefit from greater sequence complexity for templates during the initial sequencing cycles. Accordingly, a stuffer region can be used to improve sequence diversity and therefore improve the overall outcome following sequencing.

In another aspect, an enhancer region, a stuffer region, or a combination thereof can serve as a key to assist in the identification of TSO elements during data analysis. For example, identification of a sequence aligned with a stuffer region can be used to identify where the location of other sequences such as a UID/UMI.

In another aspect, the methods of the present disclosure can include a heat-denaturation step. For example, heat denaturation can be implemented following a template-switching reaction to denature some or all of the dsDNA in the TNA sample.

In another aspect, the oligonucleotide primers used in the PCR amplification steps can be designed to be complementary to sequences located in intronic regions to ensure only DNA is amplified.

In another aspect, a method according to the present disclosure can include a terminal transferase step with ddNTP after the DNA template switching step. For example, ddATP can be added to the product of the template switching reaction in excess along with Taq DNA polymerase. This would result in the addition of a terminator to all blunt ds DNA molecules and exclude them from subsequent reactions targeting RNA. The use of ddNTPs prevents products of the first template-switching reaction are not available as templates during the second template-switching reaction targeting the RNA in the sample. This can prevent the formation of DNA derived products having multiple adapters or concatemers at the 3′ termini. Moreover, original DNA templates present in the sample that did not undergo template-switching during the first template-switching reaction are tailed with a terminator and cannot be further extended during the second template-switching reaction.

In another aspect, the methods of the present disclosure can include a nuclease treatment step. For example, a nuclease enzyme can be added to the first template-switching reaction so that both blunt dsDNA that has not undergone the template-switching reaction and ssDNA would be degraded, effectively eliminating these molecules from the second template-switching reaction. In some embodiments, a nuclease treatment step can be used in place of an alternative clean-up step between the first and second template-switching reaction. One example nuclease is E. coli Exonuclease I, a 3′ to 5′ exonuclease that, when added prior to the first template-switching reaction cleanup would degrade all ssDNA in the reaction. Another example nuclease is E. coli Exo III, a 3′ to 5′ exonuclease that degrades blunt duplex dsDNA. Notably, E. coli Exo III will not digest protruding 3′ overhangs on dsDNA which could be generated by melting or removing the TSO-A.

In yet another aspect, a TSO can be designed to include a sequencing platform specific adapter sequence.

In one aspect, the methods of the present disclosure can further include the use of a capture moiety. Examples of capture moieties include biotin and desthiobiotin. In one approach, dNTPs are labeled with a capture so that, when the template-switching reaction occurs, a capture moiety is incorporated into the first nucleic acid product derived from the dsDNA template or the second nucleic acid product derived from the RNA template. The resulting labeled product can be captured using streptavidin beads. The use of capture moieties further enables the recovery of oligonucleotide primers for generating first primer extension products from RNA (e.g., RNA-specific, randomer, or oligo dT primers) by incorporating a capture moiety into the oligonucleotide primers.

The use of capture moieties additionally facilitates subsequent clean-up or purification steps. In one example, blunt dsDNA that had not undergone template-switching would be removed from the reaction during the streptavidin clean-up step prior to the second template-switching reaction, thereby reducing carryover of blunt dsDNA into the second template-switching reaction. In another aspect, if desthiobiotin labeled dNTP's are used, biotin can be added to subsequent PCR reactions to facilitate the release any template molecules attached to a capture surface such as a streptavidin coated bead.

In one embodiment of the present disclosure, A nucleic acid sample includes at least one double-stranded DNA is combined with a reverse transcriptase, a first template-switch oligo, a first mixture of dNTPs excluding at least one dNTP selected from dATP, dCTP, dGTP, and dTTP, and a 2′,3′ dideoxynucleotides (ddNTP). In one aspect, the ddNTP includes the nucleobase excluded from the first mixture of dNTPs. In performing a first template-switching reaction, the DNA template is replicated by the reverse transcriptase until the ddNTP is reached. The termination point can be controlled by choosing where the complementary base for the ddNTP is located in the first template-switch oligo. Notably, any priming off of RNA templates that may be preset in the nucleic acid sample would be halted when the ddNTP is incorporated, thereby limiting template-switching to the DNA portion of the nucleic acid sample.

In some embodiments, the ddNTP is labeled with a capture moiety. For example, the ddNTP can be labeled with biotin or desthiobiotin. During the first template-switching reaction, when the ddNTP is incorporated at the complementary position in the first template-switch oligo template, the reaction is terminated. Notably, the complementary base can be positioned within the first template-switch oligo at a defined position. The resulting product would incorporate a capture moiety (in this case, a single biotin or desthiobiotin) at the 3′ terminus of the first nucleic acid product resulting from the template-switching reaction. The first nucleic acid product can subsequently be recovered using, for example, streptavidin beads. To achieve recovery of RNA derived products resulting from a template-switching reaction according to the present disclosure, an oligonucleotide primer (e.g., a sequence specific primer, a randomer or and an oligo dT primer) can be labeled with or otherwise include at least one capture moiety. In one aspect, the use of a capture moiety can enable both purification between template-switching reactions and other downstream processing steps.

The schematic flow charts shown in the Figures are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed in the Figures are provided to explain the logical steps of the method and are understood not to limit the scope of the method. Although various arrow types and line types may be employed, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present invention is presented in several varying embodiments in the following description with reference to the Figures, in which like numbers represent the same or similar elements. Reference throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment”, and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the system. One skilled in the relevant art will recognize, however, that the system and method may both be practiced without one or more of the specific details, or with other methods, components, materials, and so forth. In other instances, well-known structures, materials, or operations are not shown or described in detail to avoid obscuring aspects of the invention. Accordingly, the foregoing description is meant to be exemplary, and does not limit the scope of present inventive concepts.

Each reference identified in the present application is herein incorporated by reference in its entirety.

1-62. (canceled)

63. A method comprising:
(a) combining into a first reaction mixture:
(i) a nucleic acid sample comprising at least one double-stranded DNA, wherein the double-stranded DNA has a first strand and a second strand, wherein the second strand is at least partially complementary to the first strand, wherein each of the first strand and the second strand have a 5′ end and a 3′ end,
(ii) a first reverse transcriptase,
(iii) a first template-switching oligonucleotide, wherein the first template-switching oligonucleotide excludes at least one nucleotide having a nucleobase selected from adenosine, cytosine, guanine, and thymine, and
(iv) a first mixture of dNTPs, wherein the first mixture excludes at least one dNTP selected from dATP, dCTP, dGTP, and dTTP, wherein the at least one dNTP excluded from the mixture of dNTPs is complementary to the at least one nucleotide excluded from the first template switching oligonucleotide; and
(b) performing a first template-switching reaction with the first reaction mixture, wherein performing the first template switching reaction comprises the following steps:
(i) adding at least one non-templated nucleotide to at least one of the 3′ ends of the double-stranded DNA with the reverse transcriptase, thereby forming a non-templated 3′ overhang on the double stranded DNA,
(ii) annealing the first template-switching oligonucleotide to the non-templated 3′ overhang of the double-stranded DNA. and
(iii) extending the non-templated 3′ overhang of the double stranded DNA with the reverse transcriptase, thereby forming a first nucleic acid product, wherein the first nucleic acid product comprises the double-stranded DNA having at least one extended 3′ end complementary to the first template-switching oligonucleotide.

64. The method of claim 63, wherein the nucleic acid sample further comprises at least one RNA, wherein the RNA has a 5′ end and a 3′ end.

65. The method of claim 63, further comprising the following steps:
(c) combining into a second reaction mixture:
(i) the first nucleic acid product,
(ii) the RNA,
(iii) a second reverse transcriptase,
(iv) a second template-switching oligonucleotide, and
(v) a second mixture of dNTPs; and
(d) performing a second template-switching reaction with the second reaction mixture, wherein performing the second template switching reaction comprises the following steps:
(i) synthesizing a polynucleotide complementary to the RNA with the second reverse transcriptase, thereby forming a first primer extension product complementary to at least a portion of the RNA;
(ii) adding at least one non-templated nucleotide to at least one of the 3′ end of the first primer extension product with the second reverse transcriptase, thereby forming a non-templated 3′ overhang on the first primer extension product;
(iii) annealing the second template-switching oligonucleotide to the non-templated 3′ overhang of the first primer extension product; and
(iv) extending the non-templated 3′ overhang of the first primer extension product with the second reverse transcriptase, thereby forming a second nucleic acid product, wherein the second nucleic acid product comprises the first primer extension product having an extended 3′ end complementary to the second template-switching oligonucleotide.

66. The method of claim 65, further comprising:
combining into the second reaction mixture, a first oligonucleotide primer having a 3′ end complementary to the RNA; and
wherein performing the second template-switching reaction with the second reaction mixture further comprises the steps of:
annealing the 3′ end of the first oligonucleotide primer to the RNA, and
extending the first oligonucleotide primer with the second reverse transcriptase, thereby forming the first primer extension product.

67. The method of claim 65, wherein the nucleotide sequence of the first template-switching oligonucleotide differs from the nucleotide sequence of the second template-switching oligonucleotide by at least one nucleotide.

68. The method of claim 64, wherein the first template-switching reaction is incapable of forming a by-product comprising a complement of at least a portion of the RNA having a 3′ end complementary to the first template-switching oligonucleotide.

69. The method of claim 63, further comprising terminating at least one of the 3′ ends of the first nucleic acid product.

70. The method of claim 69, wherein the step of terminating comprises incorporating a dideoxynucleotide at the at least one 3′ end of the first nucleic acid product.

71. The method of claim 63, wherein the first template-switching oligonucleotide includes at least one of a universal adapter sequence, a sample identified sequence, and a molecular identifier sequence.

72. The method of claim 65, wherein the second template-switching oligonucleotide includes at least one of a universal adapter sequence, a sample identified sequence, and a molecular identifier sequence.

73. The method of claim 65, wherein the method further comprises a step of purifying the nucleic acid sample comprising the first nucleic acid product and the RNA, prior to the step of combining into the second reaction mixture.

74. A method comprising:
(a) combining into a first reaction mixture:
(i) a nucleic acid sample comprising at least one double-stranded DNA, wherein the double-stranded DNA has a first strand and a second strand, wherein the second strand is at least partially complementary to the first strand, wherein each of the first strand and the second strand have a 5′ end and a 3′ end,
(ii) a reverse transcriptase,
(iii) a first template-switching oligonucleotide having a 5′ domain and a 3′ domain, wherein the 3′ domain of the first template-switching oligonucleotide excludes at least one nucleotide having a nucleobase selected from adenosine, cytosine, guanine, and thymine,
(iv) a first mixture of dNTPs, wherein the first mixture of dNTPs excludes at least one dNTP selected from dATP, dCTP, dGTP, and dTTP, wherein the at least one dNTP excluded from the mixture of dNTPs is complementary to the at least one nucleotide excluded from the 3′ domain of the first template-switching oligonucleotide, and
(v) a ddNTP complementary to the at least one nucleotide excluded from the 3′ domain of the first template-switching; and
(b) performing a first template-switching reaction with the first reaction mixture, wherein the performing a first template-switching reaction comprises the following steps:
(i) adding at least one non-templated nucleotide to at least one of the 3′ ends of the double-stranded DNA with the reverse transcriptase, thereby forming a non-templated 3′ overhang on the double-stranded DNA,
(ii) annealing the first template-switching oligonucleotide to the non-templated 3′ overhang of the double-stranded DNA, and
(iii) extending the non-templated 3′ overhang of the double-stranded DNA with the reverse transcriptase, thereby forming a first nucleic acid product, wherein the first nucleic acid product comprises the double-stranded DNA having at least one extended 3′ end complementary to the 3′ domain of the first template-switching oligonucleotide.

75. The method of claim 74, wherein the nucleic acid sample further comprises at least one RNA, wherein the RNA has a 5′ end and a 3′ end.

76. The method of claim 75, further comprising the following steps:
(a) combining into a second reaction mixture the following:
(i) the first nucleic acid product,
(ii) the RNA,
(iii) a second reverse transcriptase,
(iv) a second template-switching oligonucleotide, and
(v) a second mixture of dNTPs, and
(b) performing a second template-switching reaction with the second reaction mixture, comprising the following steps:
(i) synthesizing a polynucleotide complementary to the RNA with the second reverse transcriptase, thereby forming a first primer extension product complementary to at least a portion of the RNA,
(ii) adding at least one non-templated nucleotide to the 3′ end of the first primer extension product with the reverse transcriptase, thereby forming a non-templated 3′ overhang on the first primer extension product,
(iii) annealing the second template-switching oligonucleotide to the non-templated 3′ overhang of the first primer extension product, and
(iv) extending the non-templated 3′ overhang with the second reverse transcriptase, thereby forming a second nucleic acid product, wherein the second nucleic acid product comprises the first primer extension producing having an extended 3′ end complementary to the second template-switching oligonucleotide.

77. The method of claim 76, further comprising:
combining into the second reaction mixture, a first oligonucleotide primer having a 3′ end complementary to the RNA; and
wherein performing the second template-switching reaction with the second reaction mixture further comprises the steps of:
annealing the 3′ end of the first oligonucleotide primer to the RNA, and extending the first oligonucleotide primer with the second reverse transcriptase, thereby forming the first primer extension product.

78. The method of claim 76, wherein the nucleotide sequence of the first template-switching oligonucleotide differs from the nucleotide sequence of the second template-switching oligonucleotide by at least one nucleotide.

79. The method of claim 75, wherein the first template-switching reaction is incapable of forming a by-product comprising a complement of at least a portion of the RNA having a 3′ end complementary to the first template-switching oligonucleotide.

80. The method of claim 74, wherein the 5′ domain of the first template-switching oligonucleotide includes the at least one nucleotide excluded from the 3′ domain of the first template-switching oligonucleotide, and wherein the first nucleic acid product includes at least one 3′ end terminating in the ddNTP.

81. The method of claim 74, wherein the 3′ end of the first template-switching oligonucleotide includes at least one of a universal adapter sequence, a sample identified sequence, and a molecular identifier sequence.

82. The method of claim 76, wherein the second template-switching oligonucleotide includes at least one of a universal adapter sequence, a sample identified sequence, and a molecular identifier sequence.

83. The method of claim 76, wherein the method further comprises purifying the nucleic acid sample comprising the first nucleic acid product and the RNA, prior to the step of combining into the second reaction mixture.

84. The method of claim 65 or 76, wherein the second nucleic acid product includes a second target sequence, and wherein the method further comprises:
amplifying at least a portion of the second nucleic acid product in a second amplification reaction mixture including:
the second nucleic acid product,
a first primer having a 3′ end corresponding to at least a 5′ end of the second template-switching oligonucleotide, and
a second primer having a 3′ end corresponding to the second target sequence.

85. The method of claim 63 or 74, wherein the first nucleic acid product includes a first target sequence, and wherein the method further comprises:
amplifying at least a portion of the first nucleic acid product in a first amplification reaction mixture including:
the first nucleic acid product,
a first primer having a 3′ end corresponding to at least a 5′ end of the first template-switching oligonucleotide, and
a second primer having a 3′ end corresponding to the first target sequence.

86. The method of claim 63 or 74, wherein each of the 3′ ends of the first nucleic acid product comprises an extended 3′ end complementary to the first template-switching oligonucleotide.

87. The method of claim 65 or 76, wherein the second template-switching reaction is incapable of forming a by-product comprising the first nucleic acid product having a 3′ end complementary to the second template-switching oligonucleotide.

88. The method of claim 63 or 74, wherein the nucleic acid sample comprises a plurality of DNA.

89. The method of claim 63 or 74, wherein the nucleic acid sample comprises a plurality of RNA.

90. The method of claim 63 or 74, wherein the reverse transcriptase is selected from a Moloney Leukemia Virus (MMLV) transcriptase, an Avian Myeloblastosis Virus (AMV) reverse transcriptase, and a mutant thereof.

91. The method of claim 66 or 77, wherein the 3′ end of the first oligonucleotide primer is a poly-dT sequence.

92. The method of claim 66 or 77, wherein the 3′ end of the first oligonucleotide primer is a target-specific sequence.

93. The method of claim 66 or 77, wherein the 3′ end of the first oligonucleotide primer is a random sequence.

94. The method of claim 63 or 74, wherein the first template-switching oligonucleotide comprises a stuffer region.

95. The method of claim 66 or 77, wherein the second template-switching oligonucleotides comprises a stuffer region.

96. The method of claim 66 or 77, wherein the 3′ end of the first oligonucleotide primer is complementary to an exonic region of the RNA.

97. The method of claim 69, wherein the step of terminating comprises incorporating a dideoxynucleotide at the at least one 3′ end of the first nucleic acid product with a terminal transferase.

98. The method of claim 97, wherein the terminal transferase is Taq DNA polymerase.

99. The method of claim 63 or 74, wherein performing the first template-switching reaction with the first reaction mixture further comprises adding at least three non-templated nucleotides to at least one of the 3′ ends of the double-stranded DNA with reverse transcriptase.

100. The method of claim 65 or 76, wherein performing the second template-switching reaction with the second reaction mixture comprises adding at least three non-templated nucleotides to the 3′ end of the first primer extension product with the reverse transcriptase.

101. The method of claim 64 or 75, further comprising recovering the nucleic acid sample comprising the first nucleic acid product and the at least one RNA as a second nucleic acid sample.

102. The method of claim 65 or 76, wherein at least one of the first template-switching oligonucleotides comprises ribonucleotides and/or at least one of the second template-switching oligonucleotides comprises ribonucleotides.

103. The method of claim 102, further comprising contacting the at least one of the first template-switching oligonucleotides comprises ribonucleotides and/or the at least one of the second template-switching oligonucleotides comprises ribonucleotides, with a ribonuclease.

104. The method of claim 65 or 76, wherein at least one of the first template-switching oligonucleotide and/or at least one of the second template-switching oligonucleotide comprises a 5′ modification, wherein the 5′ modification is selected from a nucleotide analog, a linkage modification, a terminal modification, and a fluorescent label.

105. The method of claim 63 or 65, wherein the 3′ end of at least one of the first template-switching oligonucleotides and/or at least one of the second template switching oligonucleotides comprises a homopolymer sequence of at least three nucleotides.

106. The method of claim 105, wherein the homopolymer sequence is selected from polyriboguanosine, polyguanosine, polyribocytidine, and polycytidine.

107. The method of claim 74, wherein at least one of the first template-switching oligonucleotides and/or at least one of the second template-switching oligonucleotides comprises at least one 2′-O-Methyl nucleoside modification.

108. The method of claim 63 or 74, wherein the first template-switching oligonucleotides further excludes uracil.

109. The method of claim 63 or 74, wherein the first mixture of dNTPs further excludes dUTP.

110. The method of claim 74, wherein the 5′ domain of the first template-switching oligonucleotide comprises the at least one nucleotide excluded from the 3′ domain of the first template-switching oligonucleotide.

111. The method of claim 74 or claim 110, wherein the ddNTP further comprises a capture moiety.

112. The method of claim 111, wherein the capture moiety is selected from biotin and desthiobiotin.

113. The method of claim 63 or 74, wherein the first mixture of dNTPs includes at least one dNTP having a capture moiety.

114. The method of claim 113, wherein the capture moiety is selected from biotin and desthiobiotin.

115. The method of claim 65 or 76, wherein the second mixture of dNTPs includes at least one capture moiety.

116. The method of claim 115, wherein the capture moiety is selected from biotin and desthiobiotin.

117. The method of claim 66 or 77, wherein the first oligonucleotide primer includes at least one capture moiety.

118. The method of claim 117, wherein the capture moiety is selected from biotin and desthiobiotin.

119. A method comprising:
(a) combining into a first reaction mixture:
(i) a nucleic acid sample comprising at least one double-stranded DNA, wherein the double-stranded DNA has a first strand and a second strand, wherein the second strand is at least partially complementary to the first strand, wherein each of the first strand and the second strand have a 5′ end and a 3′ end, and at least one RNA, wherein the RNA has a first strand having a 5′ end and a 3′ end,
(ii) a reverse transcriptase,
(iii) a first template-switching oligonucleotide, wherein the first template-switching oligonucleotide excludes at least one nucleotide having a nucleobase selected from adenosine, cytosine, guanine, and thymine, and
(iv) a first mixture of dNTPs, wherein the first mixture excludes at least one dNTP selected from dATP, dCTP, dGTP, and dTTP, wherein the at least one dNTP excluded from the mixture of dNTPs is complementary to the at least one nucleotide excluded from the first template switching oligonucleotide; and
(b) performing a first template-switching reaction with the first reaction mixture, wherein performing the first template switching reaction comprises the following steps:
(i) adding at least one non-templated nucleotide to at least one of the 3′ ends of the double-stranded DNA with the reverse transcriptase, thereby forming a non-templated 3′ overhang on the double stranded DNA,
(ii) annealing the first template-switching oligonucleotide to the non-templated 3′ overhang of the double-stranded DNA. And
(iii) extending the non-templated 3′ overhang of the double-stranded DNA with the reverse transcriptase, thereby forming a first nucleic acid product, wherein the first nucleic acid product comprises the double-stranded DNA having at least one extended 3′ end complementary to the first template-switching oligonucleotide.

120. A method for carrying out a template-switching reaction on a nucleic acid sample, wherein the nucleic acid sample comprises at least one double-stranded DNA and at least one RNA, wherein the method comprises the following steps:
(a) performing a first template-switching reaction on the nucleic acid sample in the absence of at least one dNTP selected from dATP, dCTP, dGTP, and dTTP, thereby forming a first nucleic acid product, wherein the first nucleic acid product comprises the double-stranded DNA having at least one extended 3′ end complementary to a first template-switching oligonucleotide; and
(b) performing a second template-switching reaction on the nucleic acid sample, thereby forming a second nucleic acid product, wherein the second nucleic acid product comprises a first primer extension product complementary to at least a portion of the RNA, wherein the first primer extension product has an extended 3′ end complementary to the second template-switching oligonucleotide.

121. A kit for performing a template-switching reaction on a nucleic acid sample, wherein the nucleic acid sample comprises at least one double-stranded DNA and at least one RNA, wherein the kit comprises the following:
(a) a first template-switching oligonucleotide, wherein the first template-switching oligonucleotide excludes at least one nucleotide having a nucleobase selected from adenine, cytosine, guanine, and thymine; and
(b) a first mixture of dNTPs, wherein the first mixture of dNTPs excludes at least one dNTP selected from dATP, dCTP, dGTP, and dTTP, wherein the at least one dNTP excluded from the mixture of dNTPs is complementary to the at at least one nucleotide excluded from the first template-switching oligonucleotide.

122. The kit of claim 42, further comprising:
(c) a second template-switching oligonucleotide; and
(d) a second mixture of dNTPs.

123. A kit for performing a template-switching reaction on a nucleic acid sample, wherein the nucleic acid sample comprises at least one double-stranded DNA and at least one RNA, wherein the kit comprises the following:
(a) a first template-switching oligonucleotide, wherein the first template-switching oligonucleotide has a 5′ domain and a 3′ domain, wherein the 3′ domain of the first template-switching oligonucleotide excludes at least one nucleotide having a nucleobase selected from adenine, cytosine, guanine, and thymine;
(b) a first mixture of dNTPs, wherein the first mixture of dNTPs excludes at least one dNTP selected from dATP, dCTP, dGTP, and dTTP, wherein the at least one dNTP excluded from the first mixture of dNTPs is complementary to the at least one nucleotide excluded from the 3′ domain of the first template-switching oligonucleotide; and
(c) a ddNTP complementary to the at least one nucleotide excluded from the 3′ domain of the first template-switching oligonucleotide.

124. The kit of claim 123, further comprising:
(d) a second template-switching oligonucleotide; and
(e) a second mixture of dNTPs.
